(19) United States
(12) Patent Application Publication
(10) Pub. No.: US 2016/0158262 A1
(43) Pub. Date: Jun. 9, 2016

(54) METHODS OF TREATING IRRITABLE BOWEL SYNDROME

(71) Applicant: CEDARS-SINAI MEDICAL CENTER, Los Angeles, CA (US)

(72) Inventor: Mark Pimentel, Los Angeles, CA (US)

(73) Assignee: Cedars-Sinai Medical Center, Los Angeles, CA (US)

(21) Appl. No.: 14/906,351

(22) PCT Filed: Jul. 25, 2014
(86) PCT No.: PCT/US14/48156

§ 371 Date: Jan. 20, 2016

Related U.S. Application Data
(60) Provisional application No. 61/858,796, filed on Jul. 26, 2013.

(51) Int. Cl. A61K 31/7036; A61K 31/437

(52) U.S. Cl. CPC A61K 31/7036; A61K 31/437

(57) ABSTRACT

The present invention provides a method for treating irritable bowel syndrome (IBS) in a subject in need thereof. The treatment can include administering about 550 mg of rifaximin three times per day and about 500 mg of neomycin two times per day for 14 days to the subject to treat the IBS. In certain embodiments, the IBS is constipation predominant IBS (C-IBS).

### FIELD OF INVENTION

This invention relates to the treatment of irritable bowel syndrome.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Irritable bowel syndrome (IBS) is the most common gastrointestinal disorder accounting for significant health care and economic costs in gastroenterology. Traditionally, IBS is divided into 3 main subgroups including diarrhea-predominant IBS (D-IBS), mixed IBS (M-IBS) and constipation-predominant IBS (C-IBS). Despite the enormous burden, this disorder has suffered from poor funding. This has led to a slow progression of understanding the causes of this condition.

In the last decade, studies have begun to focus on gut microbes in the pathophysiology of IBS. One hypothesis incriminates acute gastroenteritis in the precipitation of IBS. Based on two meta-analyses, IBS develops in approximately 10% of subjects after acute gastroenteritis. Another hypothesis finds that small intestinal bacterial overgrowth (SIBO) contributes to the pathogenesis of IBS based upon breath testing, small bowel culture, small bowel qPCR, and response to antibiotics in non-C-IBS subjects.

Breath test studies suggest that methane production is associated with constipation and C-IBS. It has subsequently been determined that methane gas slows intestinal transit and, furthermore, constipation severity appears proportional to the degree of methane production. The organism believed to be largely responsible for methane production in humans is Methanobrevibacter smithii. This archaeon is resistant to many antibiotics and does not appear to respond readily to single antibiotic therapy. In a post-hoc analysis of a neomycin trial and a retrospective study, eradication of methane was found to be associated with an improvement in constipation. However, there remains a need in the art for improved methods of treating IBS and particularly C-IBS.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a study subject flowchart (CONSORT Flow Diagram) in accordance with various embodiments of the present invention.

FIG. 2 depicts primary endpoint of constipation severity in the 7 days following completion of therapy in accordance with various embodiments of the present invention. * denotes P=0.002 for direct comparison. However, after using a regression model to correct for baseline constipation VAS score, P=0.004.

FIG. 3 depicts constipation severity and bloating severity accordance with various embodiments of the present invention. a) Constipation severity throughout the whole study. P=0.0007 for the longitudinal comparison of groups by week in favor of neomycin and rifaximin using a linear mixed model analysis. b) Bloating severity through the whole study. P=0.020 for the longitudinal comparison of groups by week in favor of neomycin/rifaximin using a linear mixed model analysis.

FIG. 4 depicts straining severity and abdominal pain severity in accordance with various embodiments of the present invention. a) Straining severity through the whole study. P=0.017 for the longitudinal comparison of groups by week in favor of neomycin/rifaximin using a linear mixed model analysis. b) Abdominal pain severity through the whole study. P=0.53 for the longitudinal comparison of groups by week with no benefit of neomycin/rifaximin vs. neomycin alone using a linear mixed model analysis.

FIG. 5 depicts methane as a determinant of end of study constipation and straining severity in subjects treated with neomycin and rifaximinin accordance with various embodiments of the present invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N. Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, whether male or female, are intended to be including within the scope of this term.

“Rifaximin”, as used herein, includes solvates and polymorphous forms of the molecule, including, for example, Form α, Form β, Form γ, Form δ, Form δ, Form ζ, Form η, Form α-dry, Form t, Form β-1, Form β-2, Form ε-dry, mesylate form or amorphous forms of rifaximin. These forms are described in more detail, for example, in U.S. Pat. No. 7,915,275; U.S. Pat. No. 8,193,196; U.S. Pat. No. 7,045,620; WO 2009/108730; and G. C. Viscomi, et al., Cryst Eng Comm, 2008, 10, 1074-1081 (April 2008).

“Polymorphism”, as used herein, refers to the occurrence of different crystalline forms of a single compound in distinct hydrate status, e.g., a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as solubility profiles, melting point temperatures, hygroscopicity, particle shape, density, flowability, compatibility and/or x-ray diffraction peaks. The solubility of each polymorph may vary, thus, identifying the existence of pharmaceutical polymorphs is important for providing pharmaceuticals with predictable solubility profiles. Polymorphic forms of a compound can be distinguished in a laboratory by X-ray diffraction spectroscopy and by other methods such as, infrared spectrometry. For a general review of polymorphs and the pharmaceutical applications of polymorphs see G. M. Wall, Pharm Manuf. 3, 33 (1986); J. K. Haleblian and W. McCrone, J Pharm. Sci., 58, 911 (1969); and J. K. Haleblian, J. Pharm. Sci., 64, 1269 (1975).

“Durable response” as used herein includes adequate relief of symptoms after the cessation of the treatment regimen, and continuous adequate relief of symptoms after the cessation of the treatment regimen. The duration of the durable response can be, for example, 2 days, 5 days, 7 days, 10 days, 14 days, 3 weeks, 4 weeks, 5 weeks, 6 weeks, 7 weeks, 8 weeks, 3 months, 4 months, 5 months, 6 months or longer after the cessation of the treatment regimen. In some embodiments, the durable response can be at least 3 weeks or about 3 weeks after the cessation of the treatment regimen. In some embodiments, the durable response can be at least 4 weeks or about 4 weeks after the cessation of the treatment regimen. The assessment of the duration for the durable response can be measured for example, by the subject's subjective response, or a healthcare provider's or caretaker's assessment of the subject's symptoms.

As described herein, rifaximin complimented neomycin in significantly improving constipation and other symptoms of C-IBS. Importantly, we found a sustained benefit for 4 weeks following therapy including improvements in constipation, bloating and straining Finally, subjects receiving rifaximin plus neomycin in whom the methane biomarker was absent at 4 weeks after therapy had a greater improvement than subjects in whom methane remained present (>3 ppm).

There have been a number of studies examining antibiotic therapy in the treatment of IBS. In a non-selected population of IBS, neomycin was successful in improving IBS compared to placebo. However, neomycin had a poor rate of normalizing the breath test. Rifaximin, a non-absorbable antibiotic, has been found in multiple published randomized trials to demonstrate superiority over placebo in the treatment of IBS. The largest of these trials (TARGET 1 and TARGET 2) was specifically designed for non-constipated IBS subjects. In these two trials, the efficacy of rifaximin was seen after 14 days of rifaximin therapy and included significant improvements in global symptom rating, bloating, stool consistency and abdominal pain. More importantly, the treatment had a sustained effect such that these benefits lasted for 3 months following cessation of therapy.

While the basis of antibiotic therapy in IBS during the last decade mainly originates from the finding of an increased prevalence of SIBO in IBS, this derangement of gut microbiota is conventionally considered only in the differential diagnosis of subjects with diarrhea and not constipation. While it is difficult to assess the case for SIBO in C-IBS, breath test studies suggest that when methane is present, subjects tend to be constipated. It appears that the microbial organism responsible for methane production in humans is Methanobrevibacter smithii, since the level of this organism in stool is proportional to the level of methane on breath test, and the degree of constipation both subjectively and objectively. More importantly, a cause-and-effect relationship between methane and transit was suggested based on animal studies.

In a post hoc analysis of a neomycin double-blind study in treating IBS, the subset of C-IBS subjects with methane appeared to respond to neomycin with eradication of methane thereby favorably predicting improvement. Since neomycin alone appears effective in C-IBS, it was important in this study to have this as the active control group. In a more recent retrospective study, subjects receiving neomycin alone or rifaximin alone did not have a substantial benefit. A combination of neomycin and rifaximin appeared to eliminate methane in more than 80% of subjects with similar outcome in constipation symptoms. The interesting aspect of the study described herein is that despite having a non-placebo active control arm, there was still a benefit with combined therapy.

In this randomized double-blind placebo-controlled study we evaluated the effect of rifaximin and neomycin in the treatment of C-IBS subjects with methane present in their breath. Antibiotic therapy appears to be both beneficial regarding symptom improvement and have some degree of sustained response even after cessation of therapy. Interestingly, the response to antibiotic therapy in the case of C-IBS appears more robust in that with only a small number of subjects, there are marked differences. This may be due to a number of reasons. First, methane appears to be a marker for C-IBS. Having a marker may narrow the population only to those subjects with the abnormal microbes for the treatment. Secondly, combined antibiotics appear to have a more potent effect resulting in greater efficacy. Interestingly, methane is also a marker for success since eradication of methane predicted a favorable clinical response. In this study, unlike the previous retrospective response of >80% with rifaximin and neomycin, only half of subjects eradicated methane on breath test.

In conclusion, in this first randomized controlled trial of methane-positive C-IBS subjects, we found the combination of neomycin and rifaximin was superior to neomycin alone.

Embodiments of the present invention are based, at least in part, on the inventor's study comparing a treatment regimen with neomycin alone versus a treatment regimen with rifaximin and neomycin. The study is described in more detail herein.

Various embodiments of the present invention provide for a method for treating irritable bowel syndrome (IBS) in a subject in need thereof, comprising: administering a quantity of rifaximin and a quantity of neomycin to the subject to treat the IBS. In certain embodiments, the method further comprises providing a quantity of rifaximin and the quantity of neomycin and administering the quantity to the subject in need thereof.

In various embodiments, the subject is a mammalian subject. In certain embodiments, the subject is a human subject.

In various embodiments, the quantity of rifaximin is about 550 mg. In various embodiments, the quantity of neomycin is about 500 mg. In certain embodiments, the quantity of rifaximin is about 550 mg and the quantity of neomycin is about 500 mg.

In certain embodiments, the quantity of rifaximin is administered three times per day and the quantity of neomycin is administered two times per day. In certain embodiments, the quantity of rifaximin is administered three times per day for 14 days and the quantity of neomycin is administered two times per day for 14 days.

In various embodiments, the IBS is constipation predominant IBS (C-IBS). In various embodiments, the IBS is methane positive C-IBS.

In various embodiments, treating the IBS improves constipation in the subject. In various embodiments, the improvement in constipation has a durable response. In particular embodiments, the improvement in constipation has a durable response for at least 3 weeks or at least 4 weeks after cessation of the treatment regimen. In particular embodiments, the improvement in constipation has a durable response for about 3 weeks or about 4 weeks after cessation of the treatment regimen.

In various embodiments, treating the IBS improves bloating in the subject. In various embodiments, the improvement in bloating has a durable response. In particular embodiments, the improvement in bloating has a durable response for at least 3 weeks or at least 4 weeks after cessation of the treatment regimen. In particular embodiments, the improvement in bloating has a durable response for about 3 weeks or about 4 weeks after cessation of the treatment regimen.

In various embodiments, treating the IBS improves straining in the subject. In various embodiments, the improvement in straining has a durable response. In particular embodiments, the improvement in straining has a durable response for at least 3 weeks or at least 4 weeks after cessation of the treatment regimen. In particular embodiments, the improvement in straining has a durable response for about 3 weeks or about 4 weeks after cessation of the treatment regimen.

In various embodiments, the subject has been tested positive for methane. One of ordinary skill in the art will readily appreciate how to test for methane. Such methods are described, for example, in U.S. Pat. No. 6,861,053, which is hereby incorporated by reference as though fully set forth. In certain embodiments, the subject has a methane level of greater than 3 ppm on a lactulose breath test.

In various embodiments, the quantity of rifaximin and the quantity of neomycin is in one composition.

Various embodiments of the present invention provide for a method for treating irritable bowel syndrome (IBS) in a subject in need thereof, comprising: administering about 550 mg of rifaximin three times per day for 14 days and about 500 mg of neomycin two times per day for 14 days to the subject to treat the IBS. In certain embodiments, the IBS is constipation predominant IBS (C-IBS). In various embodiments, the IBS is methane positive C-IBS.

In various embodiments, the subject is a mammalian subject. In certain embodiments, the subject is a human subject.

In various embodiments, treating the IBS improves constipation in the subject. In various embodiments, the improvement in constipation has a durable response. In particular embodiments, the improvement in constipation has a durable response for at least 3 weeks or at least 4 weeks after cessation of the treatment regimen. In particular embodiments, the improvement in constipation has a durable response for about 3 weeks or about 4 weeks after cessation of the treatment regimen.

In various embodiments, treating the IBS improves bloating in the subject. In various embodiments, the improvement in bloating has a durable response. In particular embodiments, the improvement in bloating has a durable response for at least 3 weeks or at least 4 weeks after cessation of the treatment regimen. In particular embodiments, the improvement in bloating has a durable response for about 3 weeks or about 4 weeks after cessation of the treatment regimen.

In various embodiments, treating the IBS improves straining in the subject. In various embodiments, the improvement in straining has a durable response. In particular embodiments, the improvement in straining has a durable response for at least 3 weeks or at least 4 weeks after cessation of the treatment regimen. In particular embodiments, the improvement in straining has a durable response for about 3 weeks or about 4 weeks after cessation of the treatment regimen.

In various embodiments, the subject has been tested positive for methane. In certain embodiments, the subject has a methane level of greater than 3 ppm on a lactulose breath test.

In various embodiments, the present invention provides pharmaceutical compositions including a pharmaceutically acceptable excipient along with a therapeutically effective amount of rifaximin and/or neomycin. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. Typical dosages of an effective amount of rifaximin and neomycin can be about 550 mg of rifaximin three times per day for 14 days and about 500 mg neomycin two times per day for 14 days.

### Kits

The present invention is also directed to a kit to treat IBS. The kit is useful for practicing the inventive method of treating IBS and C-IBS. The kit is an assemblage of materials or components, including at least rifaximin and neomycin. Thus, in some embodiments the kit contains a composition including 42 doses of rifaximin at about 550 mg/dose and a composition including 28 doses of neomycin at about 500 mg/dose.

The exact nature of the components configured in the inventive kit depends on its intended purpose. For example, some embodiments are configured for the purpose of treating IBS or C-IBS. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to administer 500 mg of neomycin two times per day for 14 days and 550 mg of rifaximin three times per day for 14 days. Optionally, the kit also contains other useful components, such as, diluents, buffers, pharmaceutically acceptable carriers, syringes, catheters, applicators, pipetting or measuring tools or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in treatment of IBS. As used herein, the term “package” refers to a suitable solid matrix or material such as glass, plastic, paper, foil, and the like, capable of holding the individual kit components. Thus, for example, a package can have two plastic bottles used to contain suitable quantities of rifaximin and neomycin. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Summary of Study and Results

Consecutive C-IBS subjects (18-65 yrs of age) (Rome III criteria) were recruited after applying exclusion criteria. All subjects presented for a lactulose breath test after a 12 hour fast. Only subjects with methane level >3 ppm on at least 1 breath sample were enrolled. Subjects then completed a stool diary, questionnaire (100 point VAS scale for bloating, abdominal pain, constipation, diarrhea and straining) and 2 week run in phase. Subjects were then randomized to receive either neomycin 500 mg twice daily and placebo, or neomycin plus rifaximin 550 mg tid for 14 days in a double blind fashion. Questionnaires were repeated weekly during treatment and for 4 weeks following completion of treatment. On the last week of follow up, the stool diary was repeated.

Thirty two subjects were randomized/received drug (n=17 neomycin/placebo, n=15 neomycin/rifaximin). Baseline characteristics were not different between groups including baseline symptoms, gender and age. By week 2 of treatment, the constipation VAS score was significantly less severe in neo/rifax (30.7±30.7) group vs neo/plac (57.9±25.0) group (P=0.011). Similarly, bloating was significantly less severe in neo/rifax group compared to neo/plac (45.1±33.3 vs 65.1±25.9) group, (P=0.045). Constipation improvement has a durable response such that constipation remained significantly improved even 3 weeks following cessation of therapy (P<0.05). In addition, bloating and straining had durable improvement. In the neomycin and rifaximin group, the eradication of breath methane (≦3 ppm) was associated with a lower severity for constipation (26.6±19.0) compared to subjects with methane still present (67.2±32.1) (P=0.041).

Rifaximin combined with neomycin is superior to neomycin alone in the treatment of C-IBS subjects with methane. The best determinant of improvement was the eradication of methane after treatment in the neomycin/rifaximin group.

### Example 2

### Study and Results

### Methods

### Study Subjects

Consecutive patients (18-65 yrs of age) who fulfilled the Rome II criteria for C-IBS² were recruited. Patients were included if they reported <3 complete and spontaneous bowel movements (CSBMs) per week and had breath methane >3 ppm. Subjects were excluded if they had a history of diabetes, HIV, D-IBS, known renal disease, hearing difficulty, previous intestinal surgery (except appendectomy or cholecystectomy), current pregnancy or other known gastrointestinal disorder. Subjects were also excluded if they had taken an antibiotic or probiotic in the previous 30 days or were currently taking narcotics, proton pump inhibitors, tricyclic antidepressants, or medications known to alter intestinal motility. All subjects provided informed written consent and the study was approved by the institutional review board of all three study sites. Subjects who completed the study received a stipend. The study was registered with clinicaltrials.gov (NCT00945334).

### Screening Phase

All subjects with a diagnosis of C-IBS presented for a fasting breath test after a 12 hour fast. Only subjects with methane level of >3 ppm on a single breath sample were enrolled. Subjects then completed a baseline study questionnaire and a baseline audiogram. As there was a possibility of ototoxicity with neomycin, a baseline audiogram was performed; this was only repeated if subjects reported a subsequent change in hearing. Subjects were then asked to complete a daily stool diary for two weeks with weekly questionnaires for overall symptom severity. Subjects were eligible for randomization only if they were confirmed to have <3 CSBMs per week during stool diary.

### Treatment Phase

Subjects who successfully completed the screening phase were then randomized in a double-blind manner to receive identical tablets of either neomycin (500 mg twice daily) and placebo (three times daily) or neomycin twice daily and rifaximin (550 mg three times daily) for 14 days. The randomization scheme was set in blocks of 4 and each site received batches of drug in multiples of 4 to balance each study site into blocks. The allocation was concealed. The randomization was done by the drug company with a tracking list provided to the study site pharmacy in a concealed envelope. The pharmacy, investigators and patients were blinded. During the 14 days of treatment, subjects completed weekly symptom questionnaires (2 questionnaires over 2 weeks).

### Follow-Up Phase

Following completion of therapy, subjects were asked to continue with the weekly symptom questionnaires for an additional 4 weeks. During the final week of follow up, subjects were asked to repeat the 7-day stool diary followed by the final weekly questionnaire. Finally, subjects were asked to repeat a single fasting breath sample to examine for the presence of methane.

### Study Questionnaires

The baseline study questionnaire included subject demographics as well as symptom severity information. Follow-up questionnaires included a repeat evaluation of symptom severity. The following were assessed: abdominal pain, constipation, bloating, urgency, incomplete evacuation, straining and diarrhea. Severity was rated using a visual analog scale (VAS) from 0-100 mm (with 0=no symptom and 100=severe symptoms).

### Breath Testing

Subjects were asked to fast for 12 hours prior to a breath sample being obtained. Breath samples were collected via a Quintron dual bag collecting system (Quintron Instrument Company, Milwaukee, Wis.). The sampled gas was analyzed using a BreathTracker SC (Quintron Instrument Company, Milwaukee, Wis.). Output was reported as methane in parts per million (ppm) after correction for alveolar sample quality using breath CO₂ concentration.

### Outcome Measures

The primary outcome measure of this study was constipation severity as assessed on a VAS score from the first week following completion of the treatment phase. Secondary outcome measures included the severity of constipation, abdominal pain, urgency, bloating and straining over the entire study period. In addition, the eradication of methane was evaluated as a determinant of improvement at 4 weeks post therapy.

### Statistical Analyses Plan

The intention-to-treat (ITT) population included all individuals who had a complete baseline assessment and received at least a single dose of therapy in the treatment phase. Successful completion of treatment was considered if subjects took more than 75% of the study medication. Based on a power calculation, the study was intended to recruit a total of 88 subjects; however, due to slow enrollment, the study was ended early.

Baseline qualitative data and side effect rates were compared by Fisher's Exact Test. Quantitative baseline data was normal so were compared by t-Test. Data were expressed as mean±SD. The primary endpoint was also normally distributed and compared by t-test.

To examine the effect of each group over the duration of the study, a mixed longitudinal model was used. Because the VAS severity varied widely across weeks for most individuals, we considered week to be a categorical variable in the mixed model. Within-patient correlation across time was addressed using an autoregressive (first-order) model for the covariance structure. Missing data were mostly intermittent, and we assumed them to be missing at random. The normality assumption was satisfied for both groups most weeks for constipation, straining, and bloating data; however, normality was not satisfied for abdominal pain. The models were analyzed with a single covariate (baseline diarrhea, constipation, abdominal pain, or bloating severity score). The covariate models did not improve the fit and did not change the results substantively. Hence, we presented the simpler (no covariate) model results. All statistical analyses were conducted using SAS, version 9.1 (SAS Institute, Cary, N.C.). A P-value <0.05 was considered statistically significant.

### Results

### Study Population

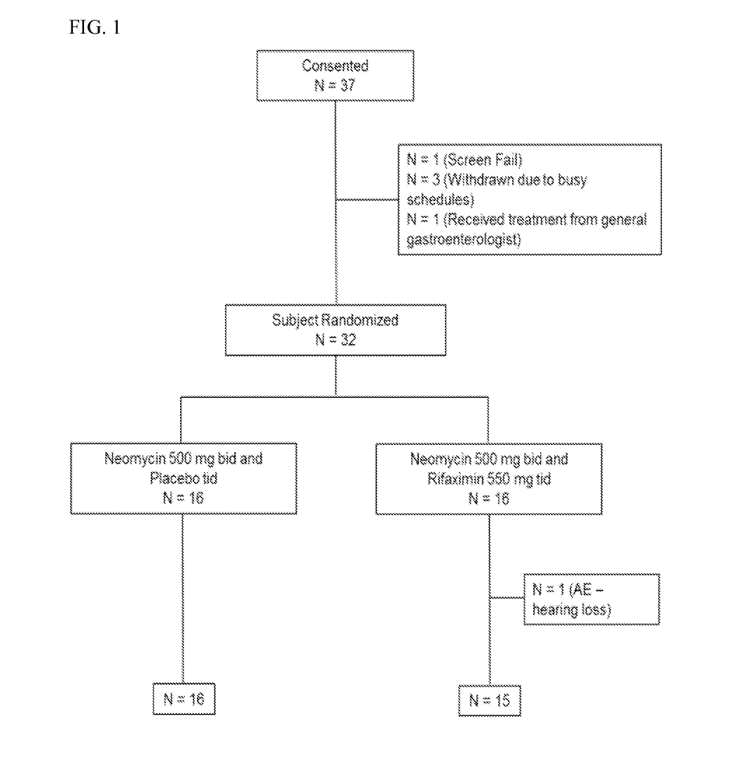

In this study, 37 C-IBS methane positive subjects were enrolled (36 from Cedars-Sinai and 1 from Mayo Clinic) (FIG. 1). Of these 37 subjects, 5 failed the screen phase and 1 subject did not have a baseline questionnaire for comparison. This left 32 subjects who entered the treatment phase and were considered part of the ITT group. These were randomized to 2 treatment groups (16 neomycin and placebo and 16 neomycin and rifaximin). The demographics and baseline characteristics were similar between these two groups (Table 1). 1 subject in the neomycin and rifaximin group withdrew from the study (see below under ‘Side Effects’), leaving 31 subjects which were included in the final ITT analysis. Of the remaining subjects, 20 returned their pill containers. All subjects took more than 75% of their medication and all but one took more than 90% of the assigned pills.

TABLE 1
Baseline demographics in ITT population.

| | Neomycin + | Neomycin + | |
|---|---|---|---|
| | Placebo | Rifaximin | P- |
| Baseline Factor | (n = 16) | (n = 15) | value |
| Age (yrs) | 40.4 ± 14.3 | 45.5 ± 16.9 | 0.37 |
| Female gender | 12 (75) | 13 (81) | 0.26 |
| [n(%)] | | | |
| Fasting Methane | 14 ± 12 | 24 ± 19 | 0.07 |
| (ppm) | | | |
| CSBM/week | 1.1 ± 0.8 | 0.6 ± 0.9 | 0.17 |
| Baseline VAS | | | |
| symptom severity (0-100) | | | |
| Constipation | 73 ± 17 | 63 ± 23 | 0.18 |
| Bloating | 76 ± 20 | 63 ± 26 | 0.14 |
| Diarrhea | 8 ± 22 | 3 ± 9 | 0.87 |
| Abdominal Pain | 44 ± 22 | 39 ± 28 | 0.62 |
| Straining | 70 ± 21 | 62 ± 28 | 0.40 |

Quantitative data expressed as mean ± SD

### Primary Outcome Measure

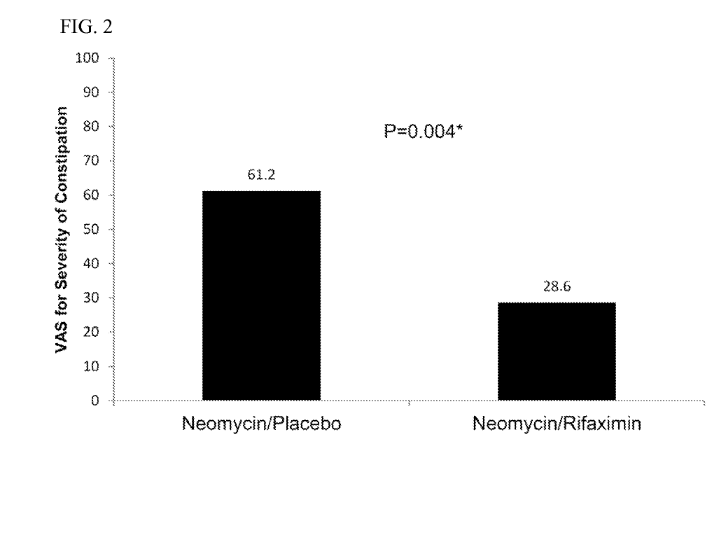

The primary endpoint was the severity of constipation in each arm at one week after completion of therapy. A lower severity score was observed for the group receiving rifaximin with neomycin. The VAS score for constipation among subjects receiving both drugs was 28.6±30.8 compared to 61.2±24.1 for neomycin alone (P=0.0020) (FIG. 2). After adjusting for baseline severity of constipation, the findings were still significant (P=0.0042).

### Secondary Outcome Measures

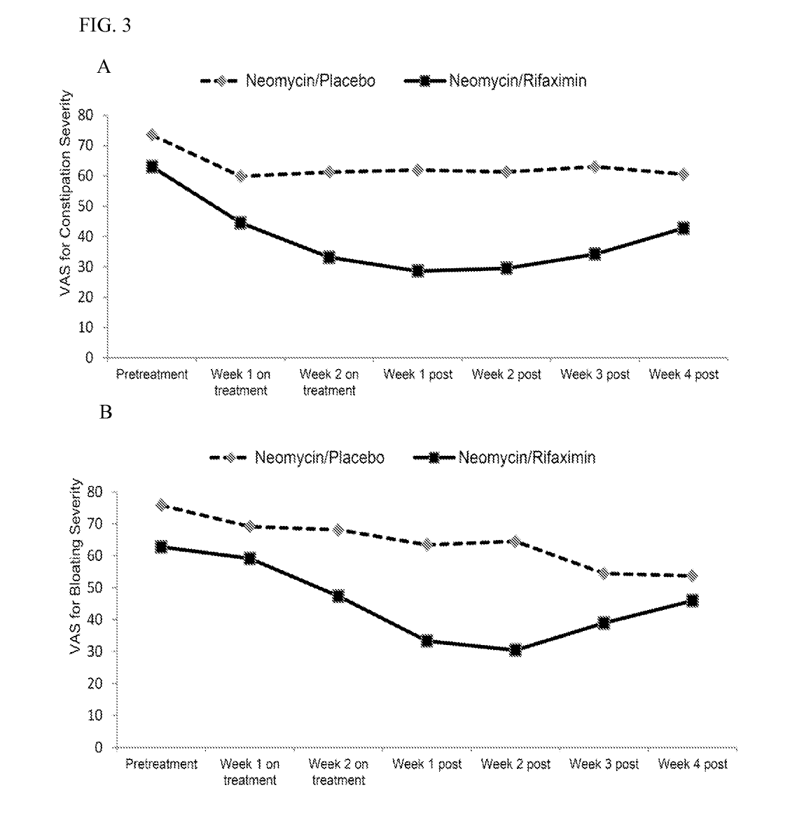

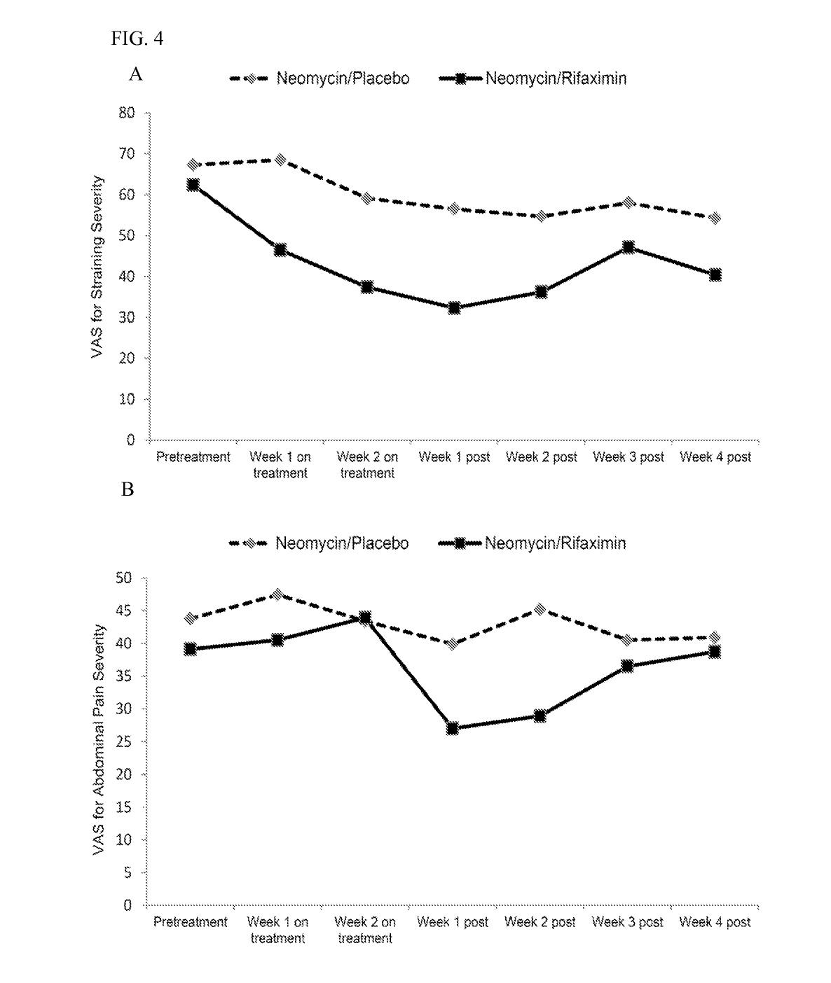

Secondary outcome measured analyses included evaluating bowel symptoms as a function of group, week, and group-by-week interaction. FIGS. 3-4 present the profiles of symptom scores by treatment arm. The profiles were essentially parallel across weeks (no interaction effect) and the mean values in the rifaximin and neomycin group were improved compared with those in the neomycin alone group, indicating the superiority of combined antibiotics across the 6 weeks of study. After treatment with rifaximin and neomycin, subjects demonstrated a sustained benefit from the treatment compared to neomycin alone for constipation (FIG. 3a), bloating (FIG. 3b) and straining (FIG. 4a), but not abdominal pain (FIG. 4b).

### Methane as a Determinant of Outcome

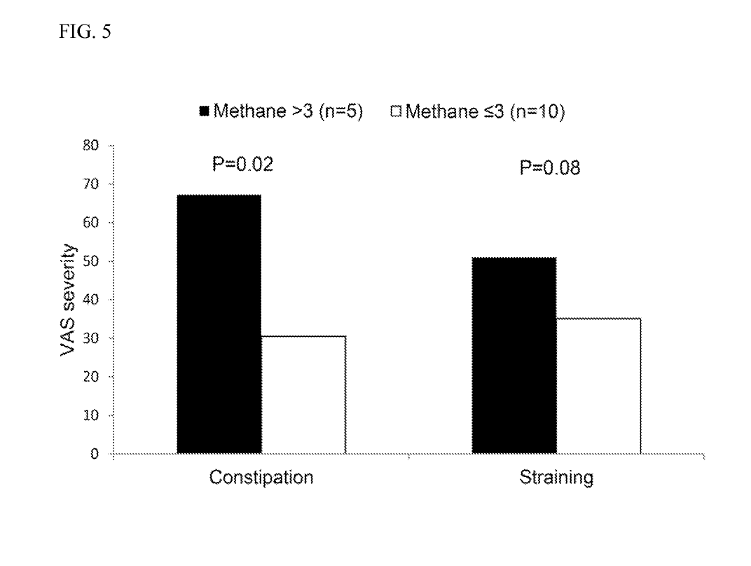

Among the 15 subjects receiving rifaximin plus neomycin, 10 subjects had methane levels ≦3 ppm following treatment. Subjects with undetectable methane based on this threshold had a lower constipation severity on the final visit than subjects who did not (FIG. 5). In the neomycin group, methane was ≦3 ppm in the final visit for 11 out of 16 subjects but there was no significant difference in constipation severity between those that did and did not have this lower methane level. The reduction in methane from baseline was greater but not significantly different between neomycin plus rifaximin (median=15 ppm drop) and neomycin alone (median=7.5 ppm drop) (P=0.45) due to small number of subjects in the study.

### Side Effects

Overall, both treatments were well tolerated by most study subjects and no side effects were reported after cessation of therapy although nausea was reported quite commonly from neomycin (Table 2). No difference was seen in terms of side effects between groups. Two adverse events were reported during the study. One male patient who received neomycin and rifaximin reported tinnitus. The subject stopped therapy, and underwent a repeat audiogram. The second audiogram demonstrated better auditory acuity than the first. Shortly after the tinnitus the subject developed an upper respiratory tract infection (URTI). The tinnitus resolved with resolution of the URTI. The second patient was a female subject in the neomycin/placebo group who felt “unwell” and stopped drug on day 12. She had begun a self-initiated weight loss program during treatment. Since she completed 75% of drug, she continued in the follow up portion of the study and completed the trial.

TABLE 2
Side effects in each arm during the two weeks of therapy.

| | Neomycin | Neomycin/ | |
|---|---|---|---|
| Side effect | [n(%)] | rifaximin [n(%)] | P-value |
| Nausea | 10 (63) | 7 (47) | 0.30 |
| Bloating and distension | 9 (56) | 7 (47) | 0.43 |
| Abdominal pain | 6 (38) | 3 (20) | 0.25 |
| Constipation | 2 (13) | 2 (13) | 0.40 |
| Diarrhea | 2 (13) | 1 (1.0) | 0.53 |
| Urgency | 2 (13) | 0 (0) | 0.26 |

Note:
Only side effects observed in more than 5% of subjects were reported.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A method for treating irritable bowel syndrome (IBS) in a subject in need thereof, comprising:
administering a quantity of rifaximin and a quantity of neomycin to the subject to treat the IBS.

2. The method of claim 1, wherein the method further comprise providing the quantity of rifaximin and the quantity of neomycin.

3. The method of claim 1, wherein the quantity of rifaximin is about 550 mg.

4. The method of claim 1, wherein the quantity of neomycin is about 500 mg.

5. The method of claim 1, wherein the quantity of rifaximin is about 550 mg and the quantity of neomycin is about 500 mg.

6. The method of claim 1, wherein administering comprises administering the quantity of rifaximin three times per day and the quantity of neomycin two times per day.

7. The method of claim 1, wherein administering comprising administering the quantity of rifaximin three times per day for 14 days and the quantity of neomycin two times per day for 14 days.

8. The method of claim 1, wherein the IBS is constipation predominant IBS (C-IBS).

9. The method of claim 1, wherein treating the IBS improves constipation, bloating, or straining in the subject.

10. The method of claim 9, wherein the constipation improvement, bloating improvement, or straining improvement has a durable response.

11. (canceled)

12. (canceled)

13. (canceled)

14. (canceled)

15. The method of claim 1, wherein the subject has tested positive for methane.

16. The method of claim 1, wherein the subject has a methane level of greater than 3 ppm on a lactulose breath test.

17. The method of claim 1, wherein the quantity of rifaximin and the quantity of neomycin is in one composition.

18. A method for treating irritable bowel syndrome (IBS) in a subject in need thereof, comprising:
administering about 550 mg of rifaximin three times per day for 14 days and about 500 mg of neomycin two times per day for 14 days to the subject to treat the IBS.

19. The method of claim 18, wherein the IBS is constipation predominant IBS (C-IBS).

20. The method of claim 18, wherein treating the IBS improves constipation in the subject.

21. The method of claim 20, wherein the constipation improvement has a durable response.

22. The method of claim 18, wherein treating the IBS improves bloating in the subject.

23. The method of claim 22, wherein the bloating improvement has a durable response.

24. The method of claim 18, treating the IBS improves straining in the subject.

25. The method of claim 24, wherein the straining improvement has a durable response.

26. The method of claim 18, wherein the subject has tested positive for methane.

27. The method of claim 18, wherein the subject has a methane level of greater than 3 ppm on a lactulose breath test.

28. A kit comprising:
42 doses of 550 mg of rifaximin;
28 doses of 550 mg of neomycin; and
instructions to administer the dose of rifaximin three times per day for 14 days and neomycin two times per day for 14 days.
